(19) United States
(12) Patent Application Publication
(10) Pub. No.: US 2018/0125042 A1
(43) Pub. Date: May 10, 2018

(54) ANIMAL MODEL HAVING HYPOPARATHYROIDISM AND METHOD FOR PRODUCING THE SAME

(71) Applicant: EWHA University-Industry Collaboration Foundation, Seoul (KR)

(72) Inventors: Han Su Kim, Seoul (KR); Soo Yeon Jung, Seoul (KR); Hae Sang Park, Gangwon-do (KR)

(21) Appl. No.: 15/789,648

(22) Filed: Oct. 20, 2017

(30) Foreign Application Priority Data
Oct. 21, 2016 (KR) 10-2016-0137560

(51) Int. Cl. A01K 67/027

(52) U.S. Cl. CPC A01K 67/0275; A01K 2267/03; C12N 2015/8527; A01K 2207/25; A01K 2227/105

(57) ABSTRACT

The present invention relates to an animal model having hypoparathyroidism and a method for producing the same. According to the present invention, the animal model having hypoparathyroidism is economical and efficient in that it can demonstrate a pathophysiology of hypoparathyroidism and maintain a survival of animals without any distortion of the pathophysiology by supplying an optimal calcium content diet.

### CROSS-REFERENCE TO RELATED APPLICATIONS

This application claims priority to KR Appl. No. 10-2016-0137560, filed Oct. 21, 2016, in the Korean Intellectual Property Office, the disclosure of which is incorporated herein its entirety by reference.

### BACKGROUND

The present invention relates to an animal model having hypoparathyroidism and a method for producing the same.

Hypoparathyroidism, which is a condition of parathyroid hormone (PTH) deficiency, is induced by various causes. The causes of hypoparathyroidism can be divided into congenital ones and acquired ones such as autoimmune diseases, iatrogenic injuries, etc. In particular, the iatrogenic injuries most commonly occur during thyroid surgery or parathyroid surgery (Rubin M R et al., Osteoporos Int. 2010; 21(11):1927-34). The PTH increases an excretion of phosphorus by kidneys, stimulates an absorption of calcium by renal tubules and small intestines, and activates osteoclasts which enhance a bone turnover. Also, the PTH activates vitamin D in kidneys. The PTH deficiency causes a loss of calcium homeostasis as well as hypocalcemia, the conditions of which lead to neurophysiological disorders including paresthesia, weakness, irritability and muscle cramping (Rejnmark L et al., Osteoporos Int. 2013; 24(5):1529-36).

An existing method for treating hypoparathyroidism involves a supplementation of calcium and vitamin D analogues, requiring patients to take oral drugs (two to eight tablets) every day. However, the existing treatment method increases a calcium level, which fails to physiologically adjust the calcium level, bone metabolism and kidney functions by means of drugs, thus increasing an incidence of complications such as fractures and nephrolithiasis (Langdahl B L et al., Bone. 1996; 18(2):103-8). Due to such limitation, there was a growing demand for a novel treatment method and a method for clinically injecting a recombinant human PTH(1-84) was introduced. However, a daily administration of the PTH differs from normal responses to physiological demands.

An animal model, which perfectly reproduces a pathophysiology of human hypoparathyroidism, is essential for verifying an efficacy of a new treatment method. An ideal animal model must be reproducible and highly cost-effective, and accurately demonstrate the human physiology and anatomy of a disease so as to make study results valid (Lelovas P P et al, Comp Med. 2008; 58(5):424-30). Now, however, there is no representative animal model having hypoparathyroidism.

A method for producing an animal model includes a surgery, radiation exposure, gene knockout and drug administration, and animals must be acclimated to and maintained under appropriate and controlled conditions so as to demonstrate a pathophysiology of a disease. In case of the animal model having hypoparathyroidism, a method for surgically removing parathyroid glands by means of microdissection and cauterization was reported (Katsumata S et al., BioFactors. 2004; 22(1-4):33-7; Chou F F et al., Hum Gene Ther. 2009; 20(11):1344-50; Liao H W et al., PLoS One. 2015; 10(7):e0133278). However, a rat's parathyroid gland is too small to be identified, thus making it difficult to accurately excise the parathyroid gland only, as well as causing a problem with an inconsistency in PTH levels after surgery (Ferreira J C et al., PLoS One. 2013; 8(11):e79721). An uncertain identification of the parathyroid gland leads to a partial parathyroidectomy or an excessive one including a thyroid gland, and such incomplete excision causes a change in PTH levels after surgery. Giving a disturbance to thyroid tissues may lead to bleeding and an excessive amount of excised tissues may cause even hypothyroidism.

Thus, in order to solve the above-mentioned problem, an animal model having hypoparathyroidism, which surgically excises only a parathyroid gland by using a 5-aminolevulinic acid (5-ALA) fluorescent identification method, was recently reported. The parathyroid gland is precisely excised without any parathyroid tissues remaining by using a fluorescent detection. According to this surgical technique, a PTH rapidly decreases to an undetectable level after surgery, thus successfully producing the animal model having hypoparathyroidism (Park Y S et al. Biomaterials. 2015; 65:140-52; Park H S et al. Eur Arch Otorhinolaryngol. 2015; 272(10):2969-77).

An ideal animal model having hypoparathyroidism must not only reproduce the pathophysiology of the disease, but also maintain the state of animals. An animal model having primary human hypoparathyroidism must prove not only a low PTH level but also a change in a balance of calcium and phosphorus. In a previous animal model using the 5-ALA fluorescent identification method, rats survived during an observation period without any signs of hypocalcaemia despite a low PTH level (Park H S et al. Eur Arch Otorhinolaryngol. 2015; 272(10):2969-77). This animal model having hypoparathyroidism differs from human hypoparathyroidism, because a low serum calcium level in humans may lead to death with an undetectably low PTH level, unless they are supplemented with additional calcium. Therefore, if a calcium-free diet (CFD) was supplied to all animals so as to exclude an effect of calcium supplied from food, all the animals died within two to three days after surgery, showing symptoms of severe hypocalcaemia, However, if calcium was supplied to the animals, a serum calcium level thereof was increased and the animals survived (Park Y S et al. Biomaterials. 2015; 65:140-52). This model successfully imitated the pathophysiology of primary hypoparathyroidism, but the CFD has problems in that it is more expensive than normal commercial feeds and causes experimental animals to die early, thus leading to high costs.

### BRIEF SUMMARY

There is a need for developing an animal model having hypoparathyroidism, which reproduces the pathophysiology of human hypoparathyroidism and is physiologically efficient and cost-effective by preventing animals from early death.

An object of the present invention is to provide an animal model having hypoparathyroidism, which can reproduce hypoparathyroidism and maintain a survival of animals, as well as a method for producing the same.

Another object of the present invention is to provide a method for screening a therapeutic substance for hypoparathyroidism by using the animal model having hypoparathyroidism.

According to the present invention, an animal model having hypoparathyroidism is economical and efficient in that it can demonstrate a pathophysiology of hypoparathyroidism and maintain a survival of animals without any distortion of the pathophysiology by supplying an optimal calcium content diet.

### BRIEF DESCRIPTION OF THE DRAWINGS/FIGURES

FIG. 1 shows a coronal plane of a rat tibia μCT-scanned by using a microcomputer, wherein a red color indicates an area of interest.

FIG. 2 shows a parathyroid gland of a rat by means of a 5-ALA fluorescent identification method of the present invention.

FIG. 3 shows results of identifying a change in a weight of a rat produced according to a producing method of the present invention.

FIG. 4 shows results of identifying a change in calcium and phosphorus levels of a rat produced according to a producing method of the present invention.

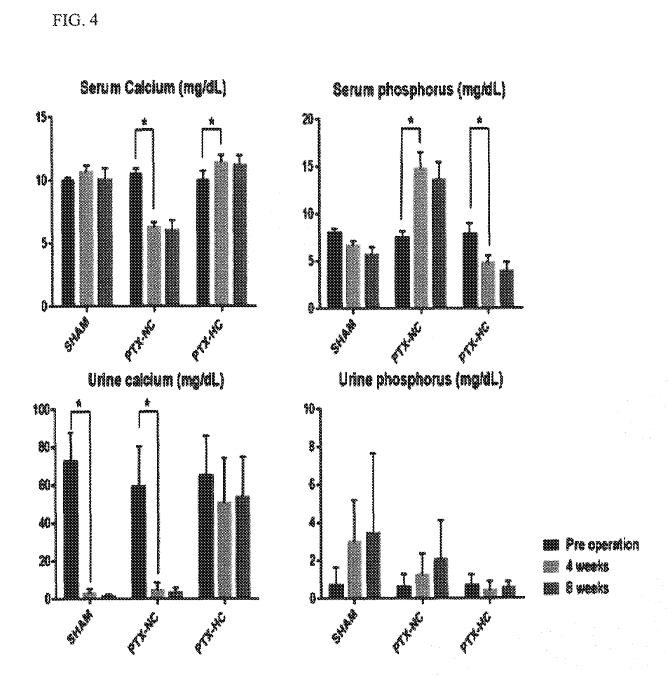

FIG. 5 shows results of identifying a stained kidney glomerulus and tubule tissue of an animal model of the present invention.

FIG. 6 shows results of identifying a change in type I collagen C-telopeptide and osteocalcin levels of a rat produced according to a producing method of the present invention.

FIG. 7 shows a μCT-scanned image of a tibia of a rat produced according to a producing method of the present invention.

### DETAILED DESCRIPTION

In order to achieve the above objects, an aspect of the present invention is a method for producing an animal model having hypoparathyroidism using a fluorescent identification method with a dietary calcium content controlled.

In particular, the present invention provides a method for producing an animal model having hypoparathyroidism, wherein the method comprises:

(a) parathyroidectomizing an experimental animal; and

(b) providing a calcium content-controlled diet to the parathyroidectomized experimental animal.

The present inventors tried to develop the animal model having hypoparathyroidism, which can be reproducible and highly cost-effective, and precisely demonstrate a physiology and anatomy of hypoparathyroidism so as to make study results valid. In result, in case of supplying the calcium content-controlled diet to the parathyroidectomized animal by using the fluorescent identification method, an optimal dietary calcium content, at which the animal model was maintained during an observation period while reproducing the pathophysiology of hypoparathyroidism, was identified.

The present invention may further comprise anesthetizing the experimental animal before the step (a). This anesthesia may be carried out according to a conventionally performed method in the art and types thereof are not limited, but may use an anesthesia method with an administration of zoletile/xylazine chloride; ketamine/xylazine; ketamine/medetomidine; ketamine/xylazine/acepromazine; sudium pentibarbital; or isoflurane, particularly zoletile/xylazine chloride.

The method for producing an animal model having hypoparathyroidism according to the present invention comprises (a) parathyroidectomizing an experimental animal.

In the present invention, the experimental animal includes all the animals, which may cause hypoparathyroidism, for example, rodents such as mice, hamsters, rats, etc., rabbits, dogs, primates, pigs, etc., excluding humans, preferably the rodents and more preferably the rats. According to one embodiment of the present invention, the experimental animal may be a Sprague-Dawley male rat, which is six to ten weeks old and weighs 260-350 g, but not limited thereto.

The method for parathyroidectomizing an experimental animal by excising parathyroid glands thereof in the step (a) may be carried out by means of all the conventionally used methods in the art, particularly micro-dissection and excising parathyroid glands by using a fluorescent identification method of the parathyroid gland.

In one embodiment of the present invention, a fluorescent identification of parathyroid glands may be performed in such a way that a red fluorescent parathyroid glands are identified under an illumination of a xenon light source with an injection of 5-aminolevulinic acid. Parathyroidectomy plays an essential role in enabling a demonstration of a pathophysiology of hypoparathyroidism in such a way that a PTH is allowed to be decreased to an undetectable level, and the parathyroid gland may be precisely excised by using the fluorescent identification method.

The present invention may further comprise measuring a serum parathyroid hormone (PTH) level after parathyroidectomy of the step (a). This measurement of the serum PTH level may be done according to a conventionally performed method in the art, preferably an enzyme-linked immunosorbent assay (ELISA), but not limited thereto.

The method for producing an animal model having hypoparathyroidism according to the present invention comprises (b) providing a calcium content-controlled diet to a parathyroidectomized experimental animal.

The method for producing an animal model of the present invention is capable of maintaining a survival of animals during an observation period by providing a calcium content-controlled diet to the animal model, wherein a calcium content of the diet may be adjusted depending on an animal, to which this model is applied, particularly 4 to 6 g/kg. In one embodiment of the present invention, the calcium content-controlled diet may be an AIN-93G containing 5 g/Kg of calcium. In case of exceeding this calcium content, for example, supplying the diet containing a high concentration of calcium (2%), a serum calcium level of the rat may increase in comparison with that of a normal control group, thus distorting the physiology of hypoparathyroidism. Also, in case of falling short of above content, there is a problem in that the experimental animal dies early.

In the step (b) of providing a calcium content-controlled diet to a parathyroidectomized experimental animal of the present invention, the diet may be further provided as a phosphorus content-controlled diet. The method is capable of maintaining a survival of animals during an observation period by providing the phosphorus content-controlled diet to the animal model, wherein a phosphorus content of the diet may be adjusted depending on an animal, to which this model is applied, particularly 1 to 2 g/kg, and more particularly 1.4 to 1.6 g/kg. In one embodiment of the present invention, the phosphorus content-controlled diet may be an AIN-93G containing 1.56 g/kg of phosphorus. A phosphorus level in blood and urine also plays an important role in evaluating a treatment effect of hypoparathyroidism. Therefore, if the phosphorus content is changed, it may have an effect on a determination of the treatment effect and cause a calcium deposit within a kidney, thus making it necessary to adjust the phosphorus content as an important factor in the animal model.

A ratio between the calcium and phosphorus contents may be 2.5 to 4.0:1, particularly 3.0 to 3.5:1.

In the present invention, the “AIN-93G,” which is a widely-used purified diet consisting of a separated protein, sugar, oil, refined vitamin and mineral, enables a constant supply of a combination of nutrients, in particular, a constant supply of minerals within a diet, in comparison with a non-purified diet, which provides a simple combination of cereals, thus the AIN-93G is appropriate for an animal experiment, wherein a body mineral level sensitively varies depending on a dietary intake. Also, the AIN-93G uses a mixture of minerals, of which a ratio between calcium and phosphorus is strictly adjusted, considering that nephrocalcinosis, etc., occurs during a long-term raising of an animal model fed with a conventionally widely used AIN-76A diet, and also has an advantage of costing less than a calcium-free diet (CFD).

Also, another aspect of the present invention is an animal model having hypoparathyroidism, which is produced by means of the aforementioned production method thereof.

The animal model having hypoparathyroidism according to the present invention is reproducible and economical, and demonstrates a pathophysiology similar to that of human hypoparathyroidism.

In case of the animal model having hypoparathyroidism of the present invention, it may be identified that hypoparathyroidism was caused by identifying at least one characteristic selected from the group consisting of (a) a decrease in a PTH level; (b) a decrease in a calcium level and an increase in a phosphorus level in serum; (c) an increase in a calcium level and a decrease in a phosphorus level in urine; (d) an increase in a bone volume; (e) an increase in a trabecular thickness; and (f) a decrease in bone resorption.

Also, another aspect of the present invention is a method for screening a therapeutic substance for hypoparathyroidism by using an animal model of the present invention.

The inventive method for screening a therapeutic substance for hypoparathyroidism can be applied to an analysis of safety and effectiveness of a therapeutic agent for hypoparathyroidism and is also appropriate for developing a novel therapeutic agent.

Particularly, the present invention provides the method for screening a therapeutic substance for hypoparathyroidism, wherein the method comprises:

(a) administering a test substance into an animal model having hypoparathyroidism of the present invention;

(b) measuring at least one screening factor selected from the group consisting of a PTH level; a calcium and phosphorus level; a bone volume; a trabecular thickness; and a bone resorption level with regard to the animal model administered with the test substance of the step (a); and

(c) selecting a substance showing a change in the factor measured above in comparison with a control group.

A “test substance,” a term used in the screening method of the present invention, means an unknown substance used in the screening to inspect whether hypoparathyroidism is improved or treated.

In the screening method, the test substance of the step (a) may be a peptide, protein, non-peptide compound, synthetic compound, fermentation product, cell extract, plant extract, animal tissue extract or plasma, etc., and the compound may be a novel compound or a known compound, particularly the one obtained from a library of synthetic or natural compounds, but not limited thereto.

The step (b) of measuring a screening factor for an animal model administered with a test substance of the step (a) comprises measuring at least one selected from the group consisting of a PTH level; a calcium and phosphorus level; a bone volume; a trabecular thickness; and a bone resorption.

According to one embodiment of the present invention, the step of measuring a screening factor may be the one of measuring a change in a serum calcium level. The measurement above may be performed by means of a conventionally used method in the art to identify a change in a serum calcium level, particularly a fully automatic biochemical analyzer, but not limited thereto.

Also, a change in a bone volume and a trabecular thickness may be measured, for example, by carrying out a micro-computed tomography (μCT).

Furthermore, the measurement of a change in a bone resorption may be performed by means of a conventionally used method thereof in a rodent animal model. According to one embodiment of the present invention, a CTX-1 level, an indicator of bone resorption activity, may be measured by using a Rat CTX-1 ELISA kit.

In the step (c) of selecting a substance showing a change in the factor measured above in comparison with a parathyroidectomized group, a determination on whether a substance is a therapeutic agent for hypoparathyroidism or not may be performed in comparison with the parathyroidectomized group, to which a test substance is not administered. For example, in case of showing an increase in a PTH level; an increase in a calcium level and a decrease in a phosphorus level in serum; a decrease in a calcium level and an increase in a phosphorus level in urine; a normalization of bone metabolism (bone volume, trabecular thickness and bone resorption) in comparison with the parathyroidectomized group, to which the test substance is not administered, the test substance is determined as a therapeutic agent for hypoparathyroidism. The therapeutic agent screened by the above-mentioned method may be very usefully used for treatment of hypoparathyroidism.

Advantages and features of the present invention as well as a method for achieving the same will be clearly understood with reference to exemplary embodiments, which will be described in detail below. However, the present invention is not limited to exemplary embodiments described herein and may be implemented in various forms. The exemplary embodiments are provided by way of example only so that a person of ordinary skill in the art can fully understand the disclosures of the present invention and the scope of the present invention. Therefore, the present invention will be defined only by the scope of the claims.

### Example 1. Preparation of Experimental Animals

Thirty male Sprague-Dawley rats (Orient Bio, Seongnam, South Korea), which are eight weeks old and weigh about 260-350 g, were used. All the animals were acclimated for at least seven days before an experiment and were allowed to have a free access to feed and water under a light-dark cycle of 12 hours. Animal care followed the Guide for the Care and Use of Laboratory Animals by the Institute of Laboratory Animal Resources and the National Institutes of Health, and the Animal Experiment Guidelines of Ewha Womans University Medical Research Institute.

### Example 2. Design of Animal Research

Animals were divided into following four groups according to their surgical procedures and calcium concentrations of diet.

1. Surgical sham (SHAM, with a parathyroid gland exposed and an incision part sutured, n=5).

2. Parathyroidectomy and calcium-free diet (PTX-FC, n=5).

3. Parathyroidectomy and normal calcium diet (PTX-NC, n=10).

4. Parathyroidectomy and high calcium diet (PTX-HC, n=10).

An AIN-93G (Research Diets, New Brunswick, N.J., USA) containing 5 g/Kg of calcium (0.5%) was used as a normal diet for the SHAM and the PTX-NC groups. Based on an AIN-93G formula, calcium-free and calcium-added formulas were produced as a customized diet, and a concentration of calcium and phosphorus containing a feed for rodents (Cargill Agri Purina, Pyongtaek, Korea) is listed in Table 1 (Park H S et al, Eur Arch Otorhinolaryngol. 2015 October; 272(10):2969-77). A dietary consumption and weight change were measured every week.

TABLE 1

| | AIN-93G | Calcium-Added | Calcium-Free | |
|---|---|---|---|---|
| | (PTX-NC, | Diet | Diet | Feed for |
| | SHAM) | (PTX-HC) | (PTX-FC) | Rodents |
| Ca (g/kg) | 5 | 20 | 0 | 11.4 |
| P (g/kg) | 1.56 | 6.24 | 1.56 | 6.1 |
| Ca:P | 3.20:1 | 3.20:1 | | 1.86:1 |
| Ratio | | | | |

In order to observe a physiology of hypoparathyroidism, levels of serum calcium, serum phosphorus, blood urea nitrogen (BUN), serum creatinine (Cr), and calcium and phosphorus in urine were measured with regard to all the groups of rats by using a fully automatic biochemical analyzer. Also, serum osteocalcin and C-telopeptide of type-I collagen (CTX-1) levels were measured by means of a rat osteocalcin (Immutopics) and rat CTX-1 ELISA kits (Cusabio, Wuhan, China), respectively. All the parameters above were evaluated before surgery; and on the fourth and eighth weeks after surgery.

All statistical analyses were performed by using an SPSS (version19) (IBM, Chicago, Ill., USA). Results were respectively represented as means±standard deviations. A repeated measure analysis of variance (RMANOVA) was used to determine a statistical significance of weight changes between the groups. A Kruskal-Wallis test was used to compare results among the three groups and a Mann-Whitney test was used to determine a statistical significance between two of the three groups. A p value<0.05 was considered significant. In each of following tables, a sign * means p-value<0.05 in the Kruskal-Wallis test among the three groups, ^{a} means a statistically significant difference between the PTX and the SHAM groups by means of the Mann-Whitney test (p-value<0.05), and ^{b} means a statistically significant difference between the PTX-NC and PTX-HC groups by the Mann-Whitney test (p-value<0.05).

### Example 3. Surgical Excision of Parathyroid Gland and Identification of PTH Levels

In order to parathyroidectomize a rat, a 5-ALA fluorescent identification method was used to remove a parathyroid gland (Park H S et al, Eur Arch Otorhinolaryngol. 2015 October; 272(10):2969-77). First of all, 5-ALA powder (Sigma-Aldrich Korea, Yongin, Korea) was suspended in a 0.9% sodium chloride solution and the resulting suspension (500 mg/kg) was administered by intraperitoneal injection to the PTX and SHAM groups. In order to prevent phototoxicity, all the animals were kept under subdued light for two hours. In two hours later, zoletile (Virbac Korea, Seoul, Korea) and xylazine chloride (Bayer Korea, Seoul, Korea) (1:1 mix, 0.1 mL/100 g) were administered by intraperitoneal injection to animals. A vertical skin incision was made at the midline of a neck, and a muscle was dissected until a trachea and a thyroid gland were exposed. Red fluorescent parathyroid glands were detected under an illumination of a xenon light (380-440 nm) source by using an ultraviolet filter designed to detect fluorescence emission at 635 nm. The parathyroid gland of the SHAM group was maintained, while two parathyroid glands of the PTX group were removed by using a cold knife. Then, hemostasis was performed by means of gauze compression and bipolar cauterization, and the incised skin was sutured by means of a non-absorbable 4-0 Ethilon® (Johnson & Johnson, New Brunswick, N.J., USA). When rats of the PTX-FC group showed a symptom of hypocalcaemia (unrestrained muscle cramping and contraction), all the five rats were euthanized within two to four days after surgery, and the rest of animals survived (eight weeks) until the study was finished.

To identify a complete removal of the parathyroid gland, a serum PTH level was measured by means of the enzyme-linked immunosorbent assay (ELISA) (Rat Bioactive Intact PTH ELISA kit, Immutopics, San Clemente, Calif., USA) on the seventh day after surgery.

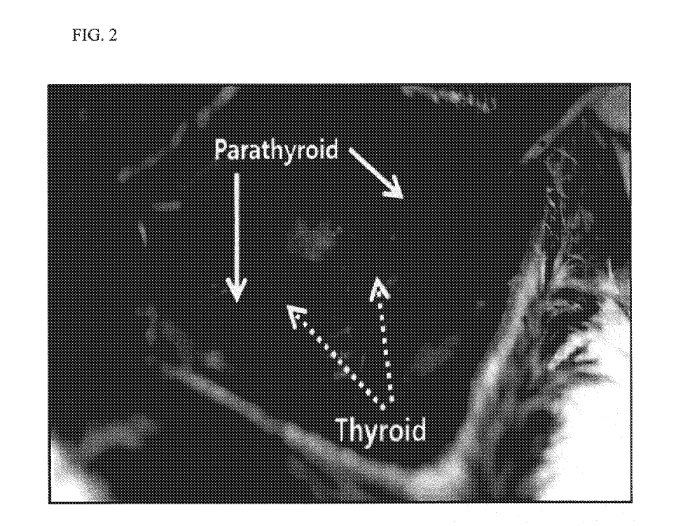

In results, the serum PTH level of the SHAM group was 88±46 pg/mL (range: 37-126 pg/mL) on the seventh day after surgery. However, the PTH of the PTX group was greatly decreased to an undetectable level in comparison with that of the SHAM group, and there was also a remarkable difference in a weight gain. On the eighth week, rats of the SHAM group weighed 599±38 g, but all the rats having survived in the PTX group weighed less than those of the SHAM group. On the eighth week, the PTX-HC group weighed more than the PTX-NC group (527±45 and 465±35 g, respectively). These results were statistically significant (FIG. 2) and the rat's food intake had no significant difference among the three groups.

### Example 4. Identification of Calcium and Phosphorus Levels

The rat's calcium and phosphorus levels were measured by using a fully automatic biochemical analyzer.

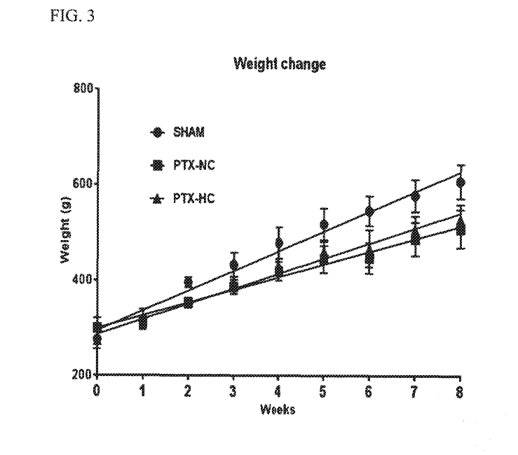

In results, comparing with the SHAM group (calcium: 10.02±0.91 mg/dL; phosphorus: 5.66±0.81 mg/dL), a serum calcium level in the PTX-NC group greatly dropped to 5.99±0.81 mg/dL and a serum phosphorus level greatly increased to 13.56±1.84 mg/dL (FIG. 3). On contrary, the PTX-HC group showed a higher serum calcium level and lower phosphorus level than the SHAM group. There was no statistically significant difference between the fourth and eight weeks.

A urine calcium level decreased in all the groups with an elapse of time, but relatively less in the PTX-HC group. The urine phosphorus level increased in the PTX-NC and SHAM groups and decreased in the PTX-HC group (FIG. 3 and Table 2). When comparing results among the groups on the eight week, the PTX-NC group showed a higher urine calcium level and a lower urine phosphorus level than the SHAM group.

TABLE 2

| | SHAM | PTX-NC | PTC-HC | P-value |
|---|---|---|---|---|
| Serum | 10.02 ± 0.89 | 5.99 ± 0.78^{ab} | 11.17 ± 0.68^{ab} | 0.000* |
| calcium | | | | |
| (mg/dL) | | | | |
| Serum | 5.66 ± 0.78 | 13.56 ± 1.76^{ab} | 3.89 ± 0.83^{b} | 0.000* |
| phosphorus | | | | |
| (mg/dL) | | | | |
| Urine | 1.31 ± 0.82 | 3.45 ± 2.61^{b} | 53.68 ± 15.37^{ab} | 0.000* |
| calcium | | | | |
| (mg/dL) | | | | |
| Urine | 3.44 ± 4.21 | 2.06 ± 1.86^{b} | 0.558 ± 0.25^{b} | 0.041* |
| phosphorus | | | | |
| (mg/dL) | | | | |

### Example 5. Evaluation of Kidney Toxicity

To evaluate an occurrence of nephrotoxicity, animals were sacrificed to obtain a kidney in eight weeks after a surgical procedure. A sample obtained was embedded in a paraffin block and stained with hematoxylin and eosin. Characteristics of kidney toxicity including tubular atrophy, interstitial fibrosis, interstitial inflammation, and glomeruli deformity were evaluated, and a presence of calcium deposit was also evaluated. A severity was graded as 0=normal histology; 1=<25%; 2=>25%, <50%; 3=>50%, <75%; and 4=>75% and results were evaluated under an optical microscope (×100 magnification).

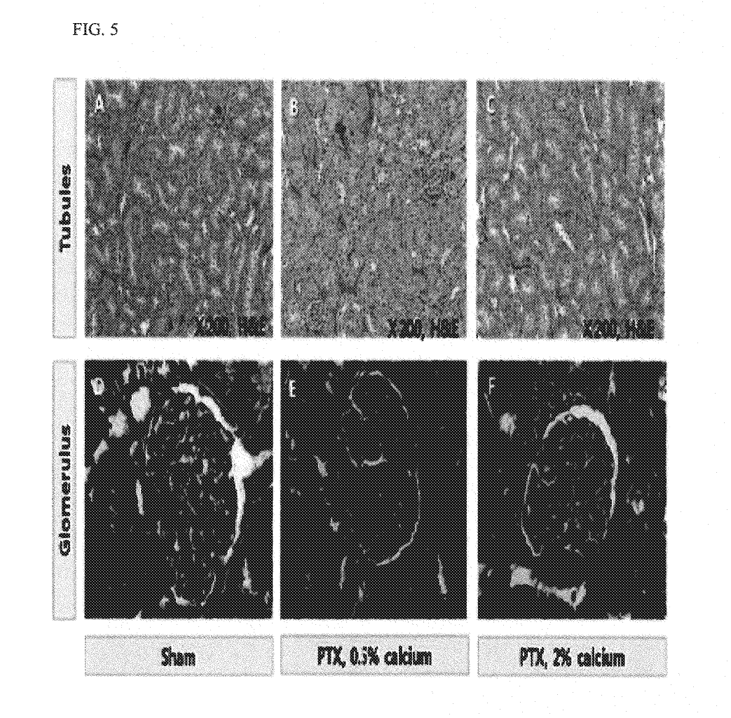

In result, it is found that the diet did not cause renal dysfunctions of PTX rats. In two months after supplying each diet, there was no change in a serum blood urea nitrogen (BUN) and creatinine levels (Table 3). The histological evaluation indicated normal tubular and glomerular structures in kidneys, and there was no sign of tubular atrophy, interstitial fibrosis, interstitial inflammation or tubular injury. Calcium phosphorus deposits were not observed on tubules or vessels (FIG. 5).

TABLE 3

| | SHAM | PTX-NC | PTC-HC | P-value |
|---|---|---|---|---|
| BUN (mg/dL) | 16.73 ± 1.97 | 15.97 ± 2.19 | 18.04 ± 3.09 | 0.080 |
| Creatinine (mg/dL) | 0.52 ± 0.09 | 0.61 ± 0.05 | 0.60 ± 0.07 | 0.054 |
| Histological grade | 0 | 0 | 0 | |
| Tubular necrosis | 0 | 0 | 0 | |
| Tubular dilation | 0 | 0 | 0 | |
| Glomerular alteration | 0 | 0 | 0 | |
| Interstitial | 0 | 0 | 0 | |
| inflammation | | | | |
| Calcium phosphorus | 0 | 0 | 0 | |
| deposit | | | | |

### Example 6. Bone Turnover Marker

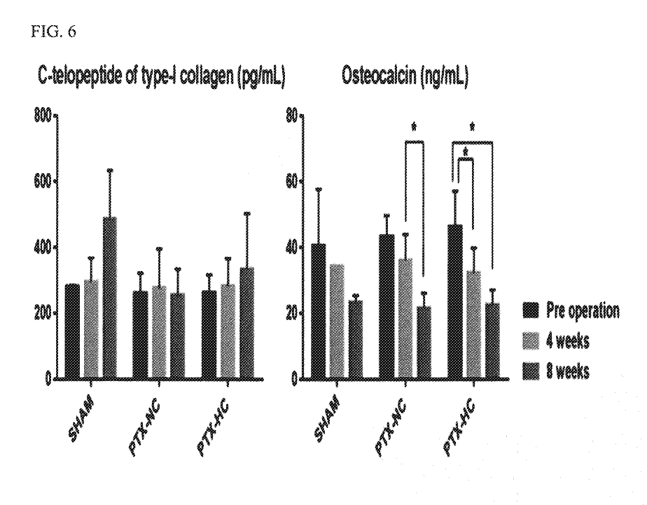

A C-telopeptide of type-I collagen (CTX-1) level, an indicator of bone resorption activity, was greatly higher in the SHAM animals than the PTX ones. The CTX-1 level of the SHAM group increased during an observation period while the CTX-1 level of the PTX group was not changed (FIG. 6). A serum osteocalcin level decreased in all the three groups with the passage of time, but there was no difference between the groups (Table 4).

TABLE 4

| | SHAM | PTX-NC | PTC-HC | P-value |
|---|---|---|---|---|
| CTX-1 | | | | |
| (pg/mL) | | | | |
| Pre | 280.27 ± 4.45 | 272.25 ± 46.97 | 262.69 ± 54.00 | |
| 8 weeks | 487.06 ± 159.97 | 256.56 ± 68.95^{a} | 304.14 ± 115.88^{a} | 0.020* |
| Osteocalcin | | | | |
| (ng/mL) | | | | |
| Pre | 40.45 ± 12.35 | 43.31 ± 6.67 | 46.20 ± 9.84 | |
| 8 weeks | 23.46 ± 1.97 | 21.67 ± 3.97 | 22.68 ± 4.20 | 0.552 |

### Example 7. Micro-Computed Tomography (CT)

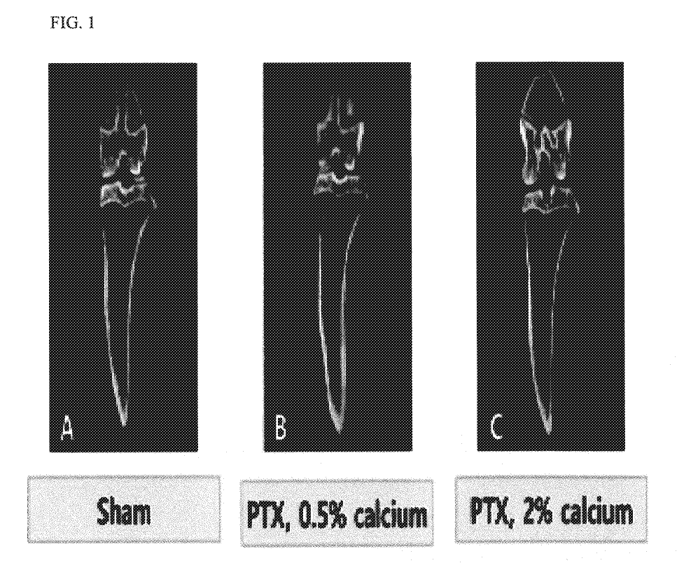

In eight weeks after surgery, μCT (NFR Polaris-G90; NanoFocusRay, Jeonju, Korea) was performed on proximal tibias of nine rats (three from SHAM, PTX-NC and PTX-HC groups, respectively). The μCT was set to 70 kVP, 50 μA, 360 views, 500 scan numbers and 512×512 reconstruction matrix. After 3D reconfiguration, a bone volume ratio, a trabecular thickness, the number of trabeculars, a trabecular sparseness and a connectivity density (Conn.D) were obtained from an area of interest (FIG. 1) by using Skyscan 1076 in vivo μCT scanner software (Skyscan, Aartselaar, Belgium).

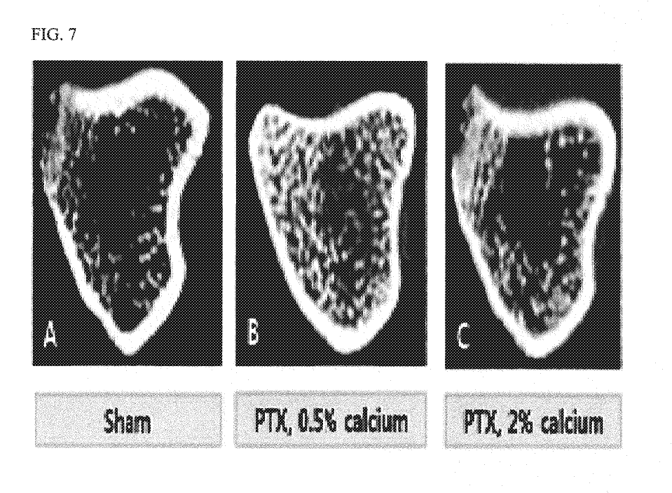

In result, the bone volume was remarkably higher in the PTX group than in the SHAM group. However, the PTX-NC group tended to have a higher bone volume ratio than the PTX-HC group, but there was no statistically significant difference among the PTX groups (Table 5). The trabecular thickness, number and sparseness did not show a significant difference between the groups. Conn. D was not different among the three groups. The μCT coronal images were shown in FIG. 7.

TABLE 5

| | SHAM | PTX-NC | PTC-HC | P-value |
|---|---|---|---|---|
| Bone | 17.791 ± 5.741 | 46.013 ± 22.29^{a} | 32.068 ± 6.644 | 0.048* |
| volume | | | | |
| ratio (%) | | | | |
| Trabecular | 0.840 ± 0.314 | 1.292 ± 0.577 | 0.821 ± 0.178 | 0.430 |
| thickness | | | | |
| (mm) | | | | |
| Number of | 0.232 ± 0.097 | 0.349 ± 0.044 | 0.392 ± 0.009 | 0.430 |
| trabeculars | | | | |
| (mm⁻¹) | | | | |
| Trabecular | 2.314 ± 1.414 | 1.918 ± 0.647 | 2.509 ± 0.221 | 0.240 |
| sparseness | | | | |
| (mm) | | | | |
| Connectivity | 0.738 ± 0.405 | 0.725 ± 0.498 | 0.862 ± 0.014 | 0.080 |
| density | | | | |
| (/mm³) | | | | |

What is claimed is:

1. A method for producing an animal model having hypoparathyroidism, wherein the method comprises:
(a) parathyroidectomizing an experimental animal excluding a human being; and
(b) providing a calcium content-controlled diet to the parathyroidectomized experimental animal.

2. The method according to claim 1, wherein the experimental animal is a rodent one.

3. The method according to claim 1, wherein the parathyroidectomy of the step (a) is performed by using a fluorescent identification method.

4. The method according to claim 3, wherein the fluorescent identification is performed with an injection of 5-aminolevulinic acid.

5. The method according to claim 1, wherein the method further comprises measuring a serum parathyroid hormone (PTH) level after the parathyroidectomy of the step (a).

6. The method according to claim 1, wherein a dietary calcium content of the step (b) is 4 to 6 g/kg.

7. The method according to claim 6, wherein the dietary calcium content is 0.5% (5 g/kg).

8. The method according to claim 1, wherein the diet of the step (b) comprises phosphorus and a phosphorus content is 1 to 2% (1 to 2 g/kg).

9. The method according to claim 8, wherein the diet is an AIN-93G.

10. The method according to claim 1, wherein a ratio between calcium and phosphorus contents of the diet is 2.5 to 4.0:1.

11. An animal model having hypoparathyroidism produced according to claim 1.

12. The animal model according to claim 11, wherein the animal model has at least one characteristic selected from the group consisting of (a) a decrease in a PTH level; (b) a decrease in a calcium level and an increase in a phosphorus level in serum; (c) an increase in a calcium level and a decrease in a phosphorus level in urine; (d) an increase in a bone volume; (e) an increase in a trabecular thickness; and (f) a decrease in bone resorption.

13. A method for screening a therapeutic substance for hypoparathyroidism, wherein the method comprises:
(a) administering a test substance into the animal model having hypoparathyroidism produced by the method of claim 1;
(b) measuring at least one screening factor selected from the group consisting of a PTH level; a calcium and phosphorus level; a bone volume; a trabecular thickness; and a bone resorption level with regard to the animal model administered with the test substance of the step (a); and
(c) selecting a substance showing a change in the factor measured in comparison with a control group.
